(19) United States
(12) Patent Application Publication
(10) Pub. No.: US 2011/0160765 A1
(43) Pub. Date: Jun. 30, 2011

(54) METHODS AND DEVICES FOR TREATMENT OF FISTULAS

(75) Inventors: Gil Y. Melmed, Los Angeles, CA (US); Simon K. Lo, Rolling Hills Estates, CA (US); Saibal Kar, Los Angeles, CA (US)

(73) Assignee: CEDARS-SINAI MEDICAL CENTER, Los Angeles, CA (US)

(21) Appl. No.: 12/937,359

(22) PCT Filed: Apr. 8, 2009
(86) PCT No.: PCT/US09/39966

§ 371 Date: Mar. 4, 2011

Related U.S. Application Data
(60) Provisional application No. 61/044,313, filed on Apr. 11, 2008.

(51) Int. Cl. A61B 17/03

(52) U.S. Cl. 606/213

(57) ABSTRACT

This invention relates to methods and devices utilizing minimally invasive techniques for digestive system fistula closure using occluder devices and various securing and sealing means. The present subject matter expands upon the breadth of current endoscopic techniques by implementing novel occluder devices and techniques intended specifically for treatment of digestive fistulas.

### RELATED APPLICATIONS

This application claims the benefit of U.S. Provisional Application Ser. No. 61/044,313 filed on Apr. 11, 2008, the entire disclosure of which is hereby incorporated by reference.

### FIELD OF THE SUBJECT MATTER

The present field of the subject matter relates to methods and devices for treatment of fistulas. Specifically, the use of minimally invasive techniques, and alternative closure devices, for the treatment of fistulas in the digestive system.

### BACKGROUND OF THE SUBJECT MATTER

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

A fistula is an abnormal connection or passageway between two epithelium-lined organs or vessels that normally do not connect. Fistulas can develop in various parts of the body, including the eye, adnexa, ear, mastoid process, circulatory system, respiratory system, digestive system, genitourinary system, the musculoskeletal system and connective tissue.

Various causes of fistula include: inflammatory bowel diseases, such as Crohn's disease and ulcerative colitis; complications from surgery, including gallbladder surgery and radiation therapy; various cancers; as well as trauma to the body which may cause perilymph fistulas and arteriovenous fistulas. In addition, obstructed labor or trauma can lead to vesicovaginal and rectovaginal fistulas.

Treatment for fistulae varies depending on the cause and extent of the fistula, but often involves surgical intervention combined with antibiotic therapy. Typically, the first step in treating a fistula is an examination by a doctor to determine the extent and “path” that the fistula takes through the tissue. In some cases, the fistula is temporarily covered; for example a fistula caused by cleft palate is often treated with a palatal obturator to delay the need for surgery until the patient reaches a more appropriate age.

Treatment for fistulae varies depending on the cause and extent of the fistula, however, the predominant and most common method for treatment of a fistula includes invasive surgery to repair the abnormality, typically combined with antibiotic therapy.

Digestive system fistulas are well-diagnosed in the medical community and encompass a broad spectrum of affected areas. As an example, gastrocolonic fistula formation is a serious complication of percutaneous endoscopic gastrostomy (“PEG”) placement [Chen Y, Ni Y H, Lai H. S. (2004) Gastrocolocutaneous fistula in a child with congenital short bowel syndrome: a rare complication of percutaneous endoscopic gastrostomy., J. Formos Med. Assoc. 103:306-10; Huang S Y, Levine M S, Raper S E. (2005) Gastrocolic fistula with migration of feeding tube into transverse colon as a complication of percutaneous endoscopic gastrostomy. AJR Am. J. Roentgenol 184:S65-6; Pitsinis V, Roberts P. (2003) Gastrocolic fistula as a complication of percutaneous endoscopic gastrostomy. Eur. J. Clin. Nutr. 57:876-8; Murphy S, Pulliam T J, Lindsay J. (1991) Delayed gastrocolic fistula following percutaneous endoscopic gastrostomy (PEG). J Am Geriatr Soc. 39:532-3; Fernandes E T, Hollabaugh R, Hixon S D, Whitington G. (1988) Late presentation of gastrocolic fistula after percutaneous gastrostomy. Gastrointest Endosc. 34:368-9; Saltzberg D M, Anand K, Juvan P, Joffe I. (1987) Colocutaneous fistula: an unusual complication of percutaneous endoscopic gastrostomy. JPEN J. Parenter. Enteral. Nutr. 11:86-7; Ponsky J L, Gauderer M W, Stellato T A. (1983) Percutaneous endoscopic gastrostomy. Review of 150 cases. Arch. Surg. 118:913-4; Strodel W E, Lemmer J, Eckhauser F, Botham M, Dent T. (1983) Early experience with endoscopic percutaneous gastrostomy. Arch. Surg. 118:449-53]. Endoscopic gastrocolonic fistula closure has been described using various techniques, but surgery is often required for definitive fistula management. However, at times surgery may not be a viable solution, especially among patients presenting a high operative risk.

Accordingly, the present subject matter addresses this deficiency in the art by disclosing a novel minimally invasive technique for digestive system fistula closure using occluder devices and various means for securing and sealing the occluder devices. As minimally invasive surgical techniques are now widely accepted and preferred, this approach should be considered for persistent fistulas as a cost reducing, safer alternative to surgical management.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts a drawing of an occluder device placed in a fistula in accordance with an embodiment of the present subject matter.

FIG. 2A depicts a computerized tomography showing a gastronomy tube inserted within the colon.

FIG. 2B depicts an image of a contrast gastrogram showing a gastrostomy tube in the colon.

FIG. 3A depicts the patent gastrocolonic fistula, as viewed from the patient's stomach, showing liquid stool in the stomach after a failed closure attempt of the fistula incorporating endoscopically placed hemoclips.

FIG. 3B depicts a magnified proximal view of the patent gastrocolonic fistula seen in FIG. 2A.

FIG. 4A depicts a three-dimensional drawing of an occluder device in accordance with an embodiment of the present subject matter.

FIG. 4B depicts a three-dimensional drawing of an occluder device in accordance with an embodiment of the present subject matter.

FIG. 5 depicts an image of a contrast gastrogram showing the successful placement of an occluder device repairing the fistula.

FIG. 6A depicts the placement of an occluder device within the fistula, as seen from the patient's stomach.

FIG. 6B depicts the placement of an occluder device within the fistula, as seen from the patient's stomach.

FIG. 7 depicts an image of a contrast gastrogram showing the successful placement of an occluder device for repair of the fistula.

### SUMMARY OF THE INVENTION

The following embodiments and aspects thereof are described and illustrated in conjunction with compositions, methods and kits meant to be exemplary and illustrative, not limiting in scope.

The present subject matter describes a method of treating a fistula comprising inserting an endoscope adjacent to a fistula and passing a guidewire through the fistula with visual assistance and guidance from the endoscope, followed by delivery of an occluder device to the fistula on the guidewire. The occluder device is therein secured in the fistula by attachment to surrounding tissue utilizing a securing device and further sealed to the tissue surrounding the fistula by a fibrant sealer.

In various embodiments, the subject matter method further comprises removing the endoscope prior to delivering the occluder device to the fistula, and re-insertion of the endoscope adjacent to the guidewire prior to delivering the occluder device to the fistula.

In another embodiment, the present subject matter method further comprises the utilization of a sizing balloon and inserting the sizing balloon into the fistula and inflating the balloon to determine fistula size prior to delivering the occluder device to the fistula.

In various embodiments, the subject matter method further comprises confirming placement of the occluder device endoscopically prior to sealing the occluder device. In yet another embodiment, the subject matter method further comprises injecting a contrast agent over the occluder device and fistula to assess fistula closure. In a particular embodiment, the subject matter further comprises performing a gastrografin enema to assess fistula closure.

In various embodiments, the subject matter method utilizes a fluoroscope for guiding occluder placement and securing and sealing the occluder device.

In various embodiments, the securing device may include a hemoclip, endoclip, triclip, resolution clip, and/or combination thereof. In another embodiment, the fibrant sealer may be cyanoacrylate or a chemical equivalent thereof.

In another embodiment the subject matter method describes a method of treating a fistula comprising imaging the fistula with a fluoroscope and passing a guidewire through the fistula with visual assistance and guidance from the fluoroscope, followed by delivery of an occluder device to the fistula on the guidewire. The occluder device is therein secured in the fistula by attachment to surrounding tissue utilizing a securing device and further sealed to the tissue surrounding the fistula by a fibrant sealer.

In various embodiments, the subject matter method further comprises inserting an endoscope adjacent to the fistula prior to delivering the occluder device to the fistula.

In another embodiment, the present subject matter method further comprises the utilization of a sizing balloon and inserting the sizing balloon into the fistula and inflating the balloon to determine fistula size prior to delivering the occluder device to the fistula.

In various embodiments, the subject matter method further comprises confirming placement of the occluder device fluoroscopically prior to sealing the occluder device. In yet another embodiment, the subject matter method further comprises injecting contrast over the occluder device and fistula to assess fistula closure. In a particular embodiment, the subject matter further comprises performing a gastrograffin enema to assess fistula closure.

In various embodiments, the subject matter method utilizes an endoscope inserted adjacent to the fistula for guiding occluder placement and securing and sealing the occluder device.

In various embodiments, the securing device may include a hemoclip, endoclip, triclip, resolution clip, and/or combination thereof. In another embodiment, the fibrant sealer may be cyanoacrylate or a chemical equivalent thereof.

The present invention further provides for a kit for the treatment of a fistula, comprising: an occluder device; at least one securing device; and a quantity of fibrant sealer for sealing the occluder device. The kit may further comprise instructions for delivering, securing and sealing the occluder device to the fistula, using at least one securing device and a therapeutically effective amount of the fibrant sealer to promote adequate treatment of the fistula. The kit may also further comprise a guidewire for delivering the occluder device to the fistula, a sizing balloon for determining the size of the fistula, and/or an endoscope for endoscopically guiding the placement, and securing and sealing the occluder device in the fistula.

In various embodiments, the securing device in the kit may be a hemoclip, endoclip, triclip, resolution clip, and/or combination thereof. In various embodiments, the fibrant sealer in the kit may be cyanoacrylate or a chemical equivalent thereof.

Other features and advantages of the invention will become apparent from the following detailed description, taken in conjunction with the accompanying drawings, which illustrate, by way of example, various features of embodiments of the invention.

### DESCRIPTION OF THE SUBJECT MATTER

All references cited herein are incorporated by reference in their entirety as though fully set forth. Unless defined otherwise, technical and scientific terms used herein have the same meaning as commonly understood by one of ordinary skill in the art to which the subject matter belongs. Singleton et al., Dictionary of Microbiology and Molecular Biology 3rd ed., J. Wiley & Sons (New York, N.Y. 2002); March, Advanced Organic Chemistry Reactions, Mechanisms and Structure 4th ed., J. Wiley & Sons (New York, N.Y. 1992); and Sambrook and Russell, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and devices similar or equivalent to those described herein, which could be used in the practice of the present subject matter. Indeed, the present subject matter is in no way limited to the methods and devices described. For purposes of the present subject matter, the following terms are defined below.

“Cardiac Septal Defect” as used herein refers to defects in the heart allowing for blood to flow between the heart's left and right chambers due to an opening in the septum.

“Fistula” as used herein refers to an abnormal connection or passageway between two epithelium-lined organs or vessels that normally do not connect. Fistulas may develop in various parts of the body, including, but not limited to: the eyes; adnexa; ear; mastoid process; the circulatory system; the respiratory system; the digestive system; connective tissue; the genitourinary system; and congenital malformations. In the interest of further clarification, a “Fistula' as used herein does not include a septal defect in the heart.

“Fluoroscope” as used herein refers to an imaging tool commonly used by physicians to obtain real-time moving images of the internal structures of a patient. In its simplest form, a fluoroscope consists of an x-ray source and fluorescent screen between which a patient is placed. Modern fluoroscopes couple the screen to an x-ray image intensifier and charge-coupled device video camera allowing the images to be recorded and played on a monitor.

“G-tube” as used herein refers to a gastronomy tube which is inserted through a small incision into the digestive system of a subject.

“Kit” as used herein refers to an assemblage of materials and/or compositions, including at least one of the components of the subject matter disclosed herein.

“Package” as used herein refers to a suitable solid matrix or material such as glass, plastic, paper, foil, and the like, capable of holding the individual kit components.

“Packaging material” refers to one or more physical structures used to house the contents of the kit, such as inventive devices and/or compositions and the like.

“PEG” as used herein refers to a percutaneous endoscopic gastrostomy (PEG), which is an endoscopic procedure for placing a tube into the stomach. It involves placing a tube into the stomach or small bowel through the abdominal wall.

“Occluder” as used herein refers to a device which repairs fistulas by plugging the opening. Occluders are typically configured to be used percutaneously.

“Spectroscope” as used herein refers to an optical instrument used to measure properties of light over a specific portion of the electromagnetic spectrum, typically used in spectroscopic analysis to identify materials. The variable measured is most often the light's intensity but could also, for instance, be the polarization state.

“Treatment” and “treating” as used herein refer to both therapeutic treatment and prophylactic or preventative measures, wherein the objective is to mend and/or repair fistulas created in the digestive system. Those in need of treatment include individuals who may have or have fistulas.

Disclosed herein are methods and devices for the treatment of fistulas using minimally invasive techniques. The present field of the subject matter employs methods and devices for the treatment of fistulas, incorporating occluder or closure devices, fibrant sealants, and minimally invasive techniques for the placement and securing of occluders and closure devices.

Ultimately, in one embodiment, the present subject matter approach, which has proven successful in fistula closure, involves 3 elements: (1) endoscopic placement of an occluder device that traverses the fistula and mechanically blocks the exchange of biological fluids and content between the two epithelium-lined organs or vessels; (2) proper anchoring of the occluder device, incorporating clips or other mechanisms, to surrounding tissue, ensuring secured placement of the occluder device; and (3) the creation of a watertight seal with the use of a fibrant sealant injected into the fistula and around the occluder device for sealing the fistula.

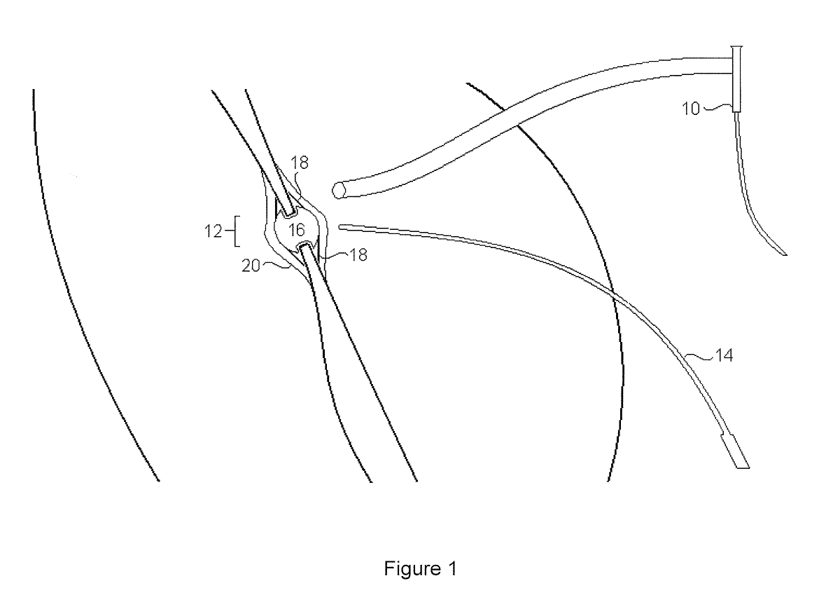

The present subject matter describes a method for treating a fistula by utilizing occluder devices, in combination with fibrant sealers and anchoring devices, to block the fistula. The use of occluder devices allows treatment of the fistula through minimally invasive techniques utilizing an endoscope. The use of an endoscope in combination with the occluder device improves upon patient recovery time, reduces medical costs and decreases morbidity. As seen in FIG. 1, the present subject matter method teaches insertion of an endoscope 10 adjacent to the fistula 12 followed by passage of a guidewire 14 to the fistula with visual assistance and guidance provided by the endoscope 10. Passage of the guidewire 14 is followed by delivery and fitting of the occluder device 16 into the fistula 12 on the guidewire 14. Once again placement and delivery of the occluder device 16 is assisted and guided by the endoscope 10. Once properly fitted in the fistula 12, the occluder device 16 is then secured in the fistula 12 by attaching the occluder device 16 to tissue surrounding the fistula 12, utilizing a securing device 18. Exemplary securing devices 18 may include a hemoclip, endoclip, triclip, resolution clip, and other devices known in the art. The occluder device 16 is thereafter further sealed to the tissue surrounding the fistula 12 by a fibrant sealer 20. The fibrant sealer 20 ensures water-tight closure of the fistula 12.

The present subject matter method may further be utilized by removing the endoscope prior to delivering the occluder device to the fistula, and re-inserting the endoscope adjacent to the guidewire prior to delivering the occluder device to the fistula. Alternative placement of the endoscope may provide a better vantage point for delivery and fitment of the occluder device, securing device and fibrant sealer. Alternatively, multiple endoscopes may be utilized to provide additional guidance and visual assistance in executing the present subject matter.

In an effort to properly measure the size of a fistula prior to placement of the occluder device, the present subject matter method further comprises a sizing balloon for insertion and inflation into the fistula. The sizing balloon may be inserted into the fistula upon the guidewire and inflated to determine fistula size. With the guidance and visual assistance of the endoscope, the present subject matter method ensures proper placement of the sizing balloon for accurate sizing of the fistula. Accurate sizing of the fistula leads to a more exact determination of the occluder device to be utilized, thus reducing incidence of ineffective fistula closure, due to improper fistula sizing.

In summary, the subject matter disclosed herein describes novel endoscopic devices and methods for fistula closure using an occluder device. The subject matter should be considered for persistent fistulas as an alternative to surgical management, particularly among patients with high operative risk. Currently available devices for cardiac septal defects were manipulated for use in fistulas, specifically digestive fistulas, and were shown to achieve successful fistula closure. Modification and retooling of these cardiac septal defect devices potentially improves long term outcome and enables better anchoring of the devices to the gastrointestinal lumen. These improvements are contemplated herein and describe the scope of the present subject matter. The present subject matter further contemplates the use of modified cardiac septal defect devices for fistula closure beyond applications established in the present subject matter, namely digestive fistulas.

The present subject matter is also directed to a kit for the treatment of fistulas in individuals possessing fistulas and/or mammals possessing fistulas. The kit is useful for practicing the inventive method of treating such conditions. The kit is an assemblage of materials and/or components, including at least one of the inventive components. Thus, in some embodiments the kit contains an occluder device and/or a composition capable of sealing said occluder device and/or an anchoring device, as described above.

The exact nature of the components configured in the inventive kit depends on its intended purpose. For example, some embodiments are configured for the purpose of treating the aforementioned conditions in an individual in need of such treatment. The kit may be configured for veterinary applications, for use in treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as to treat fistulas in individuals possessing fistulas and/or mammals possessing fistulas. Optionally, the kit may also contain other useful components such as diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable way that preserves their operability and utility. For example, the components can be in dissolved, dehydrated, or lyophilized form; they can be provided at room, refrigerated or frozen temperatures. The components are typically contained in suitable packaging material(s). The packaging material is constructed by well known methods, preferably to provide a sterile, contaminant-free environment. The packaging materials employed in the kit are those customarily utilized in the medical industry. Thus, for example, a package can be a glass vial used to contain suitable quantities of an inventive composition containing a fibrant sealant. The packaging material generally has an external label which indicates the contents and/or purpose of the kit and/or its components.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

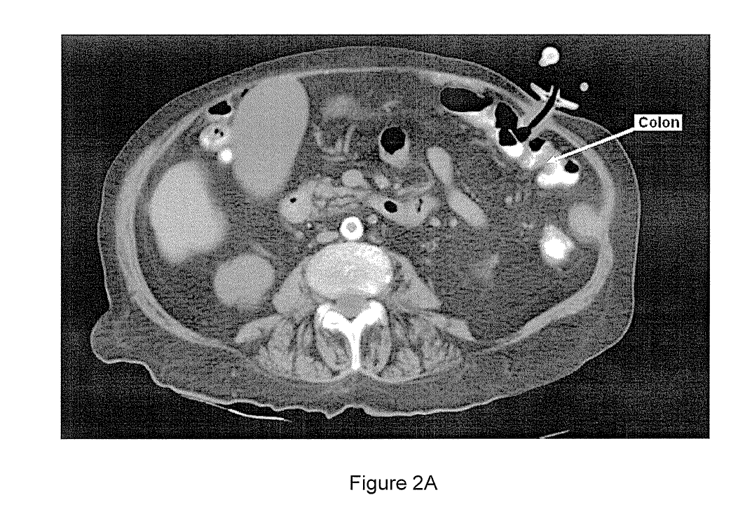

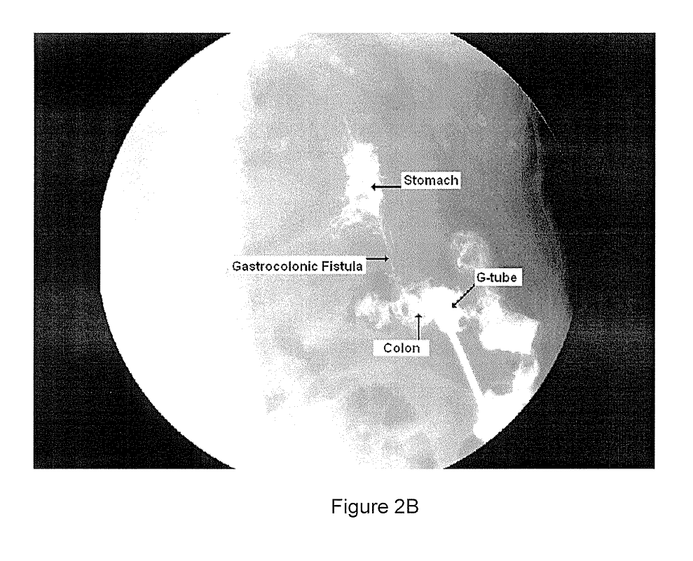

An 82 year old woman (“subject”) underwent what was thought to be an uneventful percutanous endoscopic gastrostomy tube (g-tube) placement. She presented approximately one year later with diarrhea and feculent vomiting. A contrast gastrogram of the subject, via g-tube, revealed a 1.5 cm gastrocolic fistula tract, whereby the internal bumper of the feeding tube was in the distal transverse colon (see FIG. 2A and FIG. 2B). Presumably, at the time of g-tube placement, the feeding tube had been inadvertently inserted into the stomach through the transverse colon. Over time, the internal bumper was likely inadvertently pulled back through the matured fistula tract and into the Subject's colon.

The subject was scheduled for surgical repair of the fistula, but developed cardiopulmonary compromise thought to pose an unacceptable peri-operative risk. The subject was placed on total parenteral nutrition, but had repeated metabolic and infectious complications.

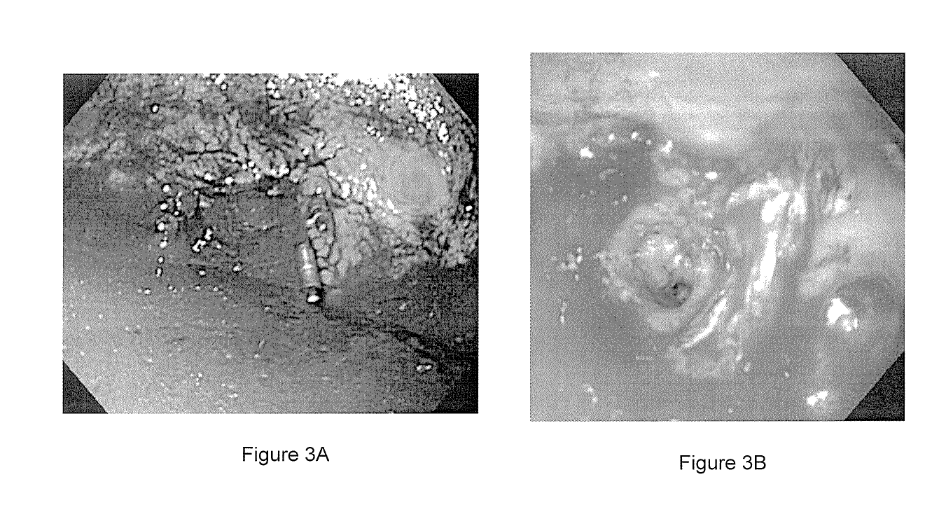

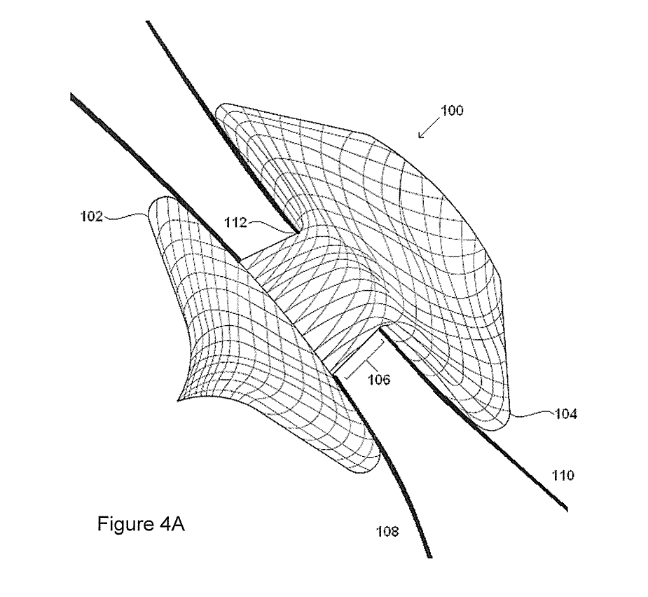

Endoscopic repair of the patent fistula was attempted a total of 4 times (see FIG. 3A and FIG. 3B). The first attempt involved cauterization of the fistula tract (to promote healing) followed by hemoclip placement with Resolution® clips deployed directly over the gastric side of the fistula opening. A new G-tube was placed and the old one removed. Over the next 2 weeks the colo-cutaneous tract spontaneously closed. However, a contrast study through the new G-tube showed persistent fistula patency.

In a second attempt at fistula closure with hemoclips, a sideviewing endoscope was passed per-rectum for optimal visualization of the distal fistula opening. However, hemoclips could not readily be deployed at the colonic side of the fistula due to angulation. The third attempt involved the use of an Endoloop circumferentially secured adjacent to the fistula edges with hemoclips followed by tightening of the Endoloop after insertion of the Surgisis® niological prosthetic mesh directly into the fistula tract. However, a gastrograffin enema obtained 2 weeks later showed continued persistent patency of the fistula tract.

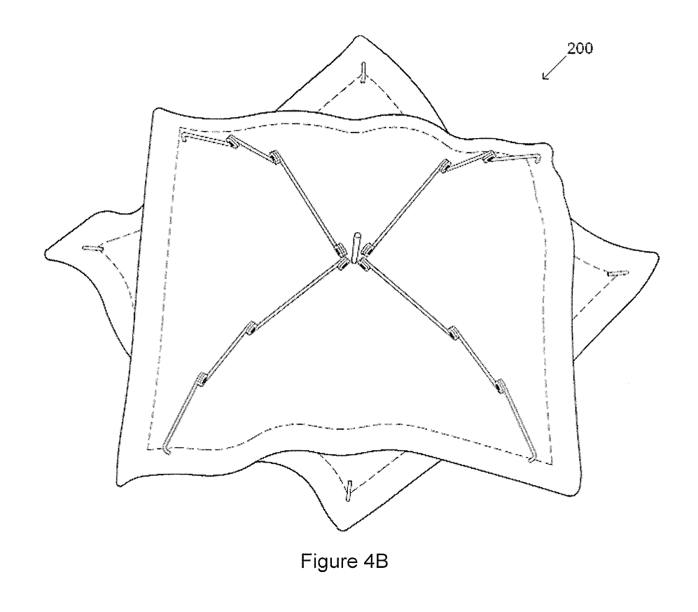

A final attempt at endoscopic gastro-colonic fistula closure using the Amplatzer Septal Occluder (“Amplatzer”) (available from AGA Medical, Plymouth, Minn.) device was successful. As illustrated in FIG. 4A, the Amplantzer device 100 is made from Nitinol wire mesh and is a self-expandable, double-disk device that is FDA-approved for closure of atrial septal defects. The distal disk 102 and proximal disk 104 are separated by a short waist 106, and within each disk 102 & 104 is a secured polyester mesh fabric. The distal disk 102 and proximal disk 104 are released sequentially through a 70 cm delivery catheter. Fore astern

In operation, the endoscope was inserted into the stomach, and a 0.035 inch super-stiff guidewire was passed endoscopically and inserted into the subject's colon through the fistula under endoscopic and fluoroscopic guidance. The endoscope was then withdrawn with the guidewire in place and was subsequently reinserted adjacent to the guidewire. The Amplatzer device delivery catheter was then exchanged over the guidewire. As seen in FIG. 4A, the Amplatzer device 100 was inserted and the distal disk 102 was deployed in the colon 108. The device was gently pulled back as the proximal disk 104 was deployed at the gastric side 110 of the fistula opening, with the short waist 106 plugging the fistula 112. Positioning of the distal disk 102 and proximal disk 104 was confirmed endoscopically and fluoroscopically.

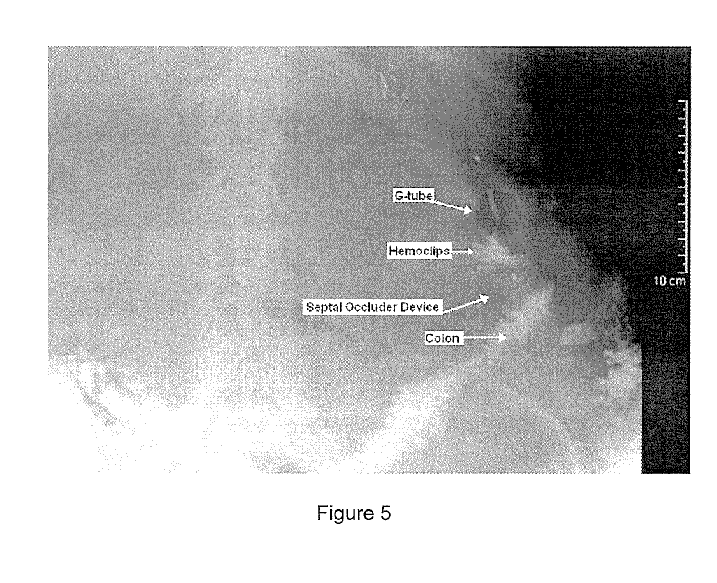

Following placement of the Amplatzer device, contrast injection over the proximal disk showed leakage through the proximal disk into the colon, indicating that the polyester membrane was not water-tight. Accordingly, 3 cc of Cyanoacrylate glue (Dermabond® available from Johnson & Johnson Corp., New Brunswick, N.J.) was injected into the proximal disk to provide a watertight seal, which was confirmed by subsequent contrast agent injection (see FIG. 5). A gastrografin enema was subsequently obtained to further confirm fistula closure. Enteral feeding was initiated 48 hrs later, and parenteral nutrition was successfully discontinued. The Amplatzer Septal Occluder device was successful for a period of approximately four months. This Amplatzer Septal Occluder device ultimately failed, likely due to tension across the wire mesh inherent in the design of a waist that was shorter in length than the length of the fistula. This tension eventually caused collapse of the Amplatzer Septal Occluder device into the colon. In accordance with the embodiment of the subject matter, similar devices with a longer waist may be well-suited for endoscopic gastrocolonic fistula closure.

### Example 2

Four months later, the same 82 year old woman once again presented with diarrhea and feculent vomiting. A contrast x-ray confirmed that the fistula thought to have been repaired was patent, and showed that the Amplatzer device had collapsed into the colon.

Due to the apparent albeit short-lived success of the concept using the Amplatzer device and accompanying endoscopic method, the decision was made to endoscopically remove the Amplatzer device and place a second occluder, the CardioSEAL® septal occluder 200, as seen in FIG. 4B (“CardioSEAL®”) (available from NMT Medical, Boston, Mass.). The CardioSEAL® lacks tension across the waist traversing the fistula tract, and based on experimentation with the Amplatzer Septal Occluder device, the CardioSEAL® was further anchored to the gastric wall using multiple clips, further securing the device. Cyanoacrylate glue was injected into the fistula after deployment to encourage a watertight seal.

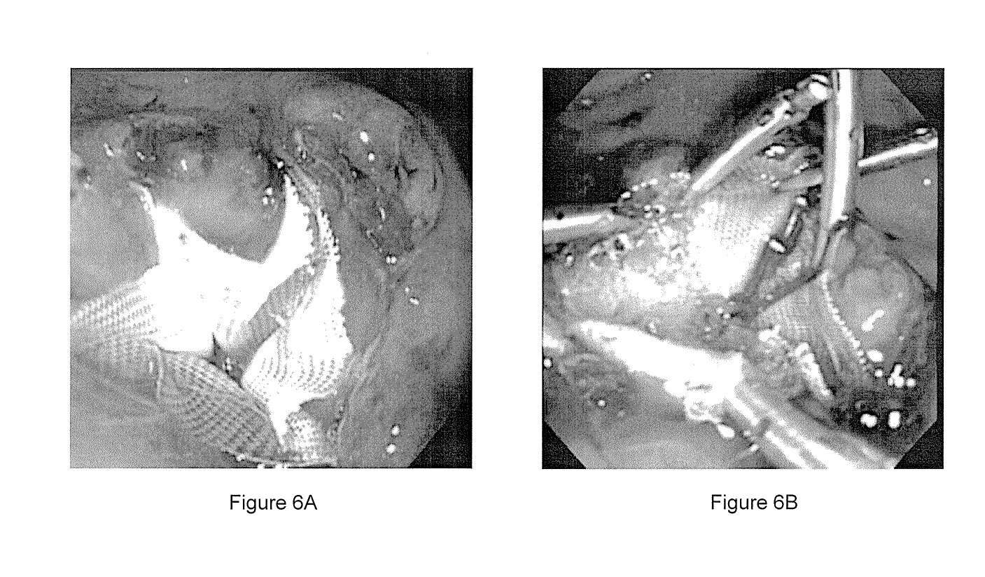

The procedure for placement of the CardioSEAL® occluder commenced with passage of a double channel upper endoscope into the Subject's stomach, and a 35 cm guidewire was advanced into the colon through the fistula. A 20 mm sizing balloon was then inserted over the guidewire and used to determine the length of the fistula (1.5 cm). The measured length of fistula lead to the necessary size of the CardioSEAL® device (33 mm) over an 11 French sheath that was exchanged over the guidewire. The CardioSEAL® device was successfully deployed under fluoroscopic guidance and multiple clips were placed at the edges of the gastric side of the deployed CardioSEAL® device in order to secure it and prevent it from migrating into the colon (see FIG. 6A and FIG. 6B). Cyanoacrylate glue (Dermabond®) was then injected through the device into the fistula tract through a needle injecting catheter.

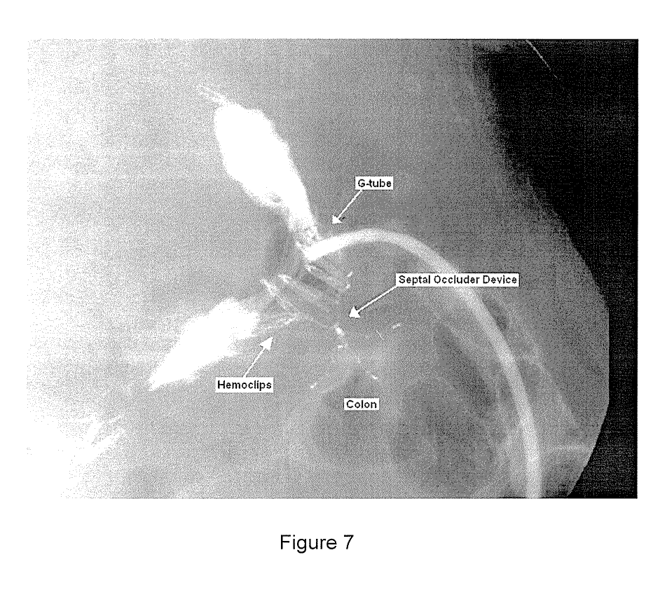

A contrast gastrogram performed one week after placement of the CardioSEAL® device showed that the fistula was no longer patent (see FIG. 7). Upper endoscopy performed about 4 months later showed that the device remained in good position, but that there was no evidence of epithelialization over the device membrane overlying the fistula tract. The patient continued to do well nearly 18 months after the CardioSEAL® device was placed, with tolerance of oral intake and no clinical evidence of fistula patency.

Methods and devices for the treatment of fistulas incorporating novel and minimally invasive techniques, an alternative occluder device, a securing device that anchors the occluder device to the adjacent tissue wall, and watertight fibrant sealants, has been achieved and disclosed. The performance of the inventive device and accompanying method have been demonstrated with success in the Examples above. The subject matter disclosed herein reduces the cost associated with treatment of fistulas, introduces viable alternatives to surgery in the treatment of fistulas, and further advances the clinical application of this technology.

While particular embodiments of the present subject matter have been shown and described, it will be obvious to those skilled in the art that, based upon the teachings herein, changes and modifications may be made without departing from this subject matter and its broader aspects and, therefore, the appended claims are to encompass within their scope all such changes and modifications as are within the true spirit and scope of this subject matter. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

What is claimed is:

1. A method of treating a fistula in an individual, comprising:
inserting an endoscope into the individual adjacent to the fistula;
passing a guidewire through the fistula utilizing the endoscope;
delivering an occluder device to the fistula on the guidewire utilizing the endoscope;
securing the occluder device in the fistula to tissue surrounding the fistula using a securing device; and
sealing the occluder device in the fistula with a fibrant sealer.

2. The method of claim 1, further comprising removing the endoscope prior to delivering the occluder device to the fistula.

3. The method of claim 2, further comprising re-inserting the endoscope adjacent to the guidewire prior to delivering the occluder device to the fistula.

4. The method of claim 1, further comprising inserting a sizing balloon into the fistula and inflating the balloon to determine fistula size prior to delivering the occluder device to the fistula.

5. The method of claim 1, further comprising confirming placement of the occluder device endoscopically prior to sealing the occluder device.

6. The method of claim 1, further comprising injecting contrast over the occluder device and fistula to assess fistula closure.

7. The method of claim 1, further comprising performing a gastrograffin enema to assess fistula closure.

8. The method of claim 1, further comprising guiding occluder placement and securing and sealing the occluder device utilizing a fluoroscope.

9. The method of claim 1, wherein the securing device is selected from the group consisting of: hemoclip, endoclip, triclip, resolution clip, and a combination thereof.

10. The method of claim 1, wherein the fibrant sealer is cyanoacrylate or a chemical equivalent thereof.

11. A method of treating a fistula, comprising:
imaging the fistula with a fluoroscope;
passing a guidewire through the fistula utilizing the fluoroscope;
delivering an occluder device to the fistula on the guidewire utilizing the fluoroscope;
securing the occluder device in the fistula to tissue surrounding the fistula using a securing device; and
sealing the occluder device in the fistula with a fibrant sealer.

12. The method of claim 11, further comprising inserting an endoscope adjacent to the fistula to guide occluder placement.

13. The method of claim 11, further comprising inserting an endoscope adjacent to the guidewire to guide occluder placement.

14. The method of claim 11, further comprising inserting a sizing balloon into the fistula and inflating the balloon to determine fistula size prior to delivering the occluder device to the fistula.

15. The method of claim 11, further comprising confirming placement of the occluder device fluoroscopically prior to sealing the occluder device.

16. The method of claim 11, further comprising injecting contrast agent over the occluder device and fistula to assess fistula closure.

17. The method of claim 11, further comprising performing a gastrograffin enema to assess fistula closure.

18. The method of claim 11, further comprising guiding occluder securing and sealing with an endoscope.

19. The method of claim 11, wherein the securing device is selected from the group consisting of: hemoclip, endoclip, triclip, resolution clip, and a combination thereof.

20. The method of claim 11, wherein the fibrant sealer is cyanoacrylate or a chemical equivalent thereof.

21. A kit for the treatment of a fistula, comprising:
an occluder device;
at least one securing device;
a quantity of fibrant sealer for sealing the occluder device; and
instructions for delivering, securing and sealing the occluder device to the fistula, using at least one securing device and a therapeutically effective amount of the fibrant sealer to promote adequate treatment of the fistula.

22. The kit of claim 21, further comprising a guidewire for delivering the occluder device to the fistula.

23. The kit of claim 21, further comprising a sizing balloon for determining the size of the fistula.

24. The kit of claim 21, further comprising an endoscope for endoscopic guidance, placement, securing, and sealing of the occluder device in the fistula.

25. The kit of claim 21, wherein the securing device is selected from the group consisting of: hemoclip, endoclip, triclip, resolution clip, and a combination thereof.

26. The kit of claim 21, wherein the fibrant sealer is cyanoacrylate or a chemical equivalent thereof.
